(19) United States
(12) Patent Application Publication
(10) Pub. No.: US 2018/0206742 A1
(43) Pub. Date: Jul. 26, 2018

(54) BLOODSTREAM DETECTING DEVICE AND BLOODSTREAM DETECTOR USED THEREIN

(71) Applicant: HAN-WEI ZHANG, New Taipei City (TW)

(72) Inventor: HAN-WEI ZHANG, New Taipei City (TW)

(21) Appl. No.: 15/872,145

(22) Filed: Jan. 16, 2018

(30) Foreign Application Priority Data
Jan. 20, 2017 (CN) 201720067799.5

(51) Int. Cl. A61B 5/026; A61B 5/00

(52) U.S. Cl. CPC A61B 5/0261; A61B 5/4064; A61B 5/6814; A61B 2560/0412; A61B 5/14551; A61B 2562/0219; A61B 2562/0238; A61B 2576/026; A61B 2560/045

(57) ABSTRACT

A bloodstream detector includes a covering sheet, light emitters, light receivers, an orientation sensor, and a signal output interface. The covering sheet includes a detection surface. The light emitters are on the covering sheet and emit lights outwardly from the detection surface. The light receivers are on the covering sheet and exposed from the detection surface to receive lights coming toward the detection surface. The orientation sensor is on the covering sheet to detect an orientation change of the bloodstream detector. The signal output interface is coupled to the light receivers and the orientation sensor to output detection signals of the light receivers and the orientation sensor.

### CROSS-REFERENCE TO RELATED APPLICATION

This non-provisional application claims priority under 35 U.S.C. § 119(a) to Patent Application No. 201720067799.5 filed in China, P.R.C. on Jan. 20, 2017, the entire contents of which are hereby incorporated by reference.

### BACKGROUND

### Technical Field

The instant disclosure relates to a detector, in particular, to a bloodstream detecting device and a bloodstream detector used therein.

### Related Art

In recent years, nuclear magnetic resonance imaging (NMRI) techniques are applied for observing brain activities to help psychiatrists in determining of conditions of the patients. However, the NMRI equipment is expensive. Moreover, noises and environments of the equipment during measurements easily make the patient feel stressful.

### SUMMARY

In view of these, one embodiment of the instant disclosure provides a bloodstream detector. The bloodstream detector comprises a covering sheet, at least one light emitter, at least one light receiver, an orientation sensor, and a signal output interface. The covering sheet comprises a detection surface. The at least one light emitter is on the covering sheet and emits lights outwardly from the detection surface. The at least one light receiver is on the covering sheet and is exposed from the detection surface to receive lights coming toward the detection surface. The orientation sensor is on the covering sheet to detect an orientation change of the bloodstream detector. The signal output interface is coupled to the at least one light receiver and the orientation sensor to output a detection signal of the at least one light receiver and a detection signal of the orientation sensor.

Another embodiment of the instant disclosure provides a bloodstream detecting device. The bloodstream detecting device comprises the aforementioned bloodstream detector and a computing device. The computing device is electrically connected to the signal output interface of the bloodstream detector to receive the detection signal of the at least one light receiver and the detection signal of the orientation sensor.

As above, the bloodstream detector of some embodiments of the instant disclosure can be manufactured with low costs and can be applied to the testees in a convenient manner. Moreover, the signals of the orientation sensor can optimize the signals of the light receiver, facilitating the reading of the signals of the light receiver.

### BRIEF DESCRIPTION OF THE DRAWINGS

The disclosure will become more fully understood from the detailed description given herein below for illustration only, and thus not limitative of the disclosure, wherein:

FIG. 1 illustrates a perspective view (1) of a bloodstream detector according to an exemplary embodiment of the instant disclosure;

FIG. 2 illustrates a perspective view (2) of the bloodstream detector;

FIG. 3 illustrates a schematic block diagram (1) of the bloodstream detector;

FIG. 4 illustrates a schematic block diagram (2) of the bloodstream detector;

FIG. 5 illustrates a schematic block diagram of a bloodstream detecting device according to an exemplary embodiment of the instant disclosure;

FIG. 6 illustrates an operational view (1) of the bloodstream detector;

FIG. 7 illustrates an operational view (2) of the bloodstream detector;

FIG. 8 illustrates an operational view of a bloodstream detector according to another embodiment of the instant disclosure; and

FIG. 9 illustrates an operational view of a bloodstream detector according to yet another embodiment of the instant disclosure.

### DETAILED DESCRIPTION

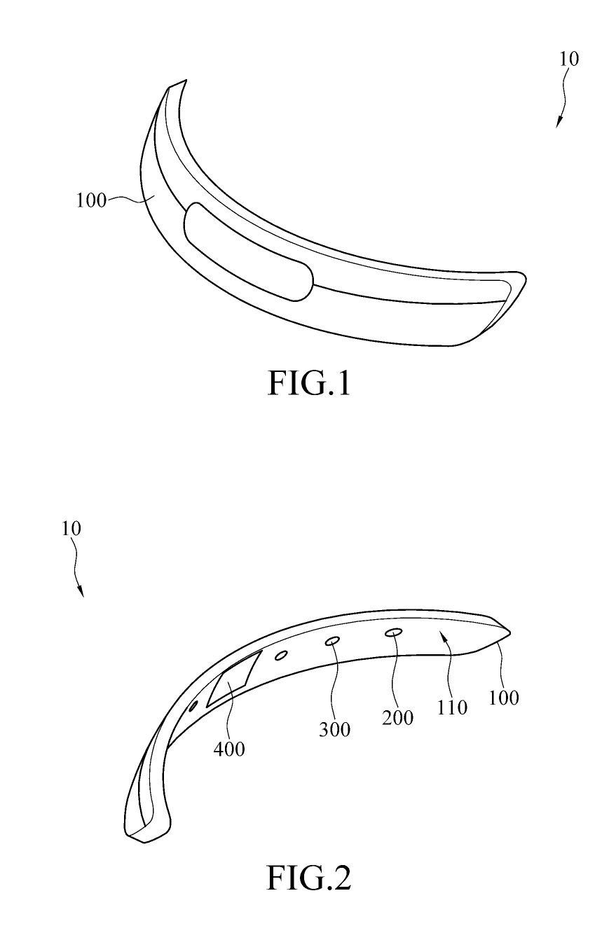

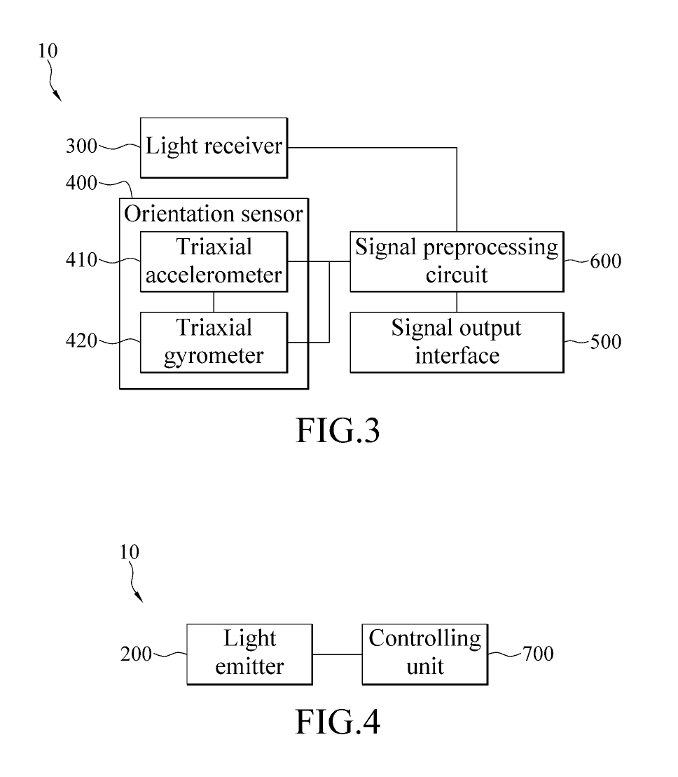

FIGS. 1 and 2 illustrate perspective views of a bloodstream detector 10 according to an exemplary embodiment of the instant disclosure from different angles. FIG. 3 illustrates a schematic block diagram (1) of the bloodstream detector 10. The bloodstream detector 10 comprises a covering sheet 100, a light emitter 200, a light receiver 300, an orientation sensor 400, and a signal output interface 500. In this embodiment, for description, the bloodstream detector 10 comprises a plurality of light emitters 200 and a plurality of light receivers 300, but embodiments are not limited thereto. In one embodiment, the bloodstream detector 10 may comprise one light emitter 200 and one light receiver 300.

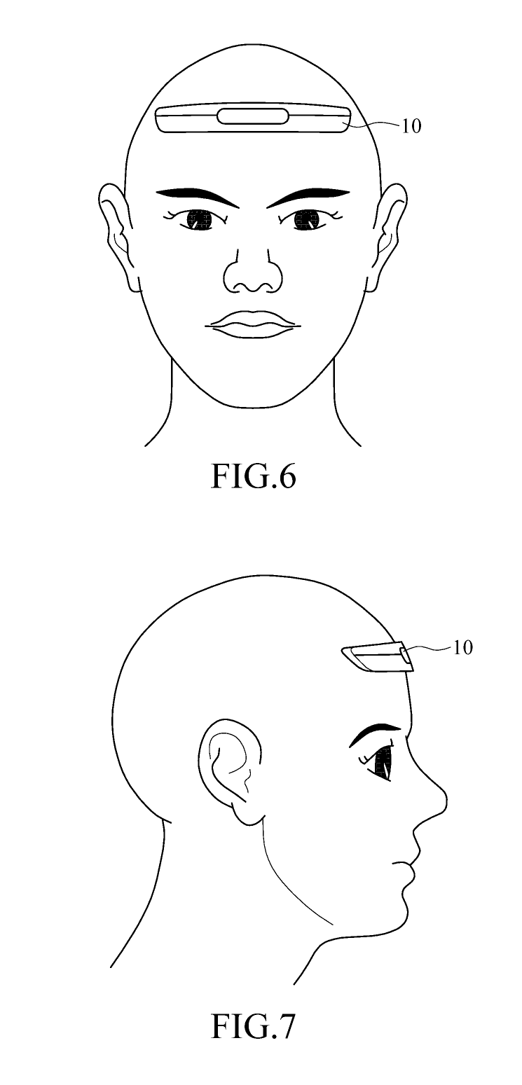

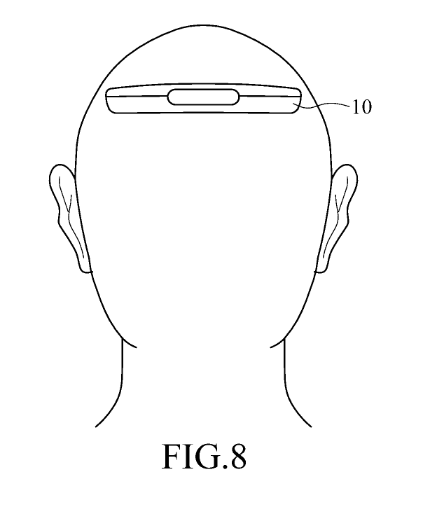

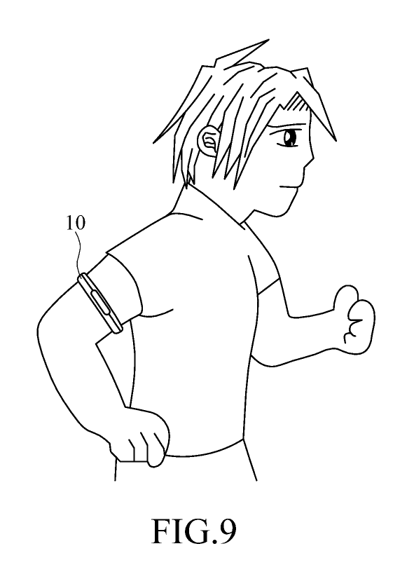

The covering sheet 100 is of an elongate shape. The covering sheet 100 comprises a detection surface 110, and the detection surface 110 is adapted for covering on a head portion of a testee. As shown in FIGS. 6 and 7, the bloodstream detector 10 is covering on the forehead of the testee, but embodiments are not limited thereto. In some embodiments, the bloodstream detector 10 may be covering on the back of the head of the testee (as shown in FIG. 8). That is, the bloodstream detector 10 may be placed on a location corresponding to the portion of the testee's head which brain activities happens. In some embodiments, the bloodstream detector 10 may be placed on other portions, such as the body or limbs of the testee, to measure the change of the oxygen content of the muscle for analyzing the properties of the testee's muscle. Hence, the test provider can realize that the testee is suitable for performing exercises requiring high muscular explosiveness or exercises requiring high muscular endurance. As shown in FIG. 9, the bloodstream detector 10 is placed on the upper arm of the testee. In some other embodiments, the bloodstream detector 10 may be placed on the thigh or the leg of the testee (not shown).

In one embodiment, the bloodstream detector 10 is fixed on the skin of the testee by gluing, but embodiments are not limited thereto. In some embodiments, the bloodstream detector 10 may be fixed on the testee by straps.

As shown in FIGS. 1 and 2, the light emitter 200 is disposed on the covering sheet 100 to emit lights outwardly from the detection surface 110. In this embodiment, in order to allow the lights permitting the skin of the testee and entering into the subcutaneous blood vessels of the testee, the wavelength of the lights emitted by the light emitter 200 is in the range of the wavelengths corresponding to red and infrared lights. The light receiver 300 is disposed on the covering sheet 100 and exposed from the detection surface 110 to receive lights coming toward the detection surface 110. In other words, when the bloodstream detector 10 covers the skin of the testee, the lights received by the light receiver 300 are the lights emitted by the light emitter 200 and reflected by the skin of the testee. In this embodiment, the light emitter 200 and the light receiver 300 are arranged in pairs. Namely, one light emitter 200 and one light receiver 300 form a group in which the light emitter 200 and the light receiver 300 are adjacently arranged, but embodiments are not limited thereto. In one embodiment, one light emitter 200 may correspond to several light receivers 300; in another embodiment, several light emitters 200 may correspond to one light receiver 300.

In one embodiment, the wavelength of the lights may be in the range between 750 nm and 860 nm. In another embodiment, the wavelength of the lights may be in the range between 800 nm and 820 nm. In yet another embodiment, the wavelength of the lights may be in the range between 750 and 770 nm, between 805 nm and 815 nm, or between 840 nm and 860 nm.

In one embodiment, the light emitter 200 outputs one of the aforementioned wavelength ranges. In another embodiment, the light emitter 200 outputs two or more of the aforementioned wavelength ranges. For instance, the light emitter 200 may emit lights having a wavelength in the range between 750 nm and 860 nm and lights having a wavelength in the range between 800 nm and 820 nm. In this embodiment, the light emitter 200 has two light sources for outputting the lights with two wavelength ranges. In one embodiment, the light emitter 200 emits the lights with two wavelength ranges at the same time. In another embodiment, the light emitter 200 emits the lights with two wavelength ranges alternately.

In one embodiment, the light emitter 200 and the light receiver 300 are aligned linearly. In one embodiment, the light emitter 200 and the light receiver 300 are aligned along a long axis passing through the middle portion of the covering sheet 100.

The orientation sensor 400 is also disposed on the covering sheet 100 for detecting an orientation change of the bloodstream detector 10. As shown in FIG. 3, the orientation sensor 400 comprises a triaxial accelerometer 410 and a triaxial gyroscope 420. The triaxial accelerometer 410 is adapted to measure the accelerations of the bloodstream detector 10 in three axes, and the triaxial gyrometer 420 is adapted to measure the angular velocities of the bloodstream detector 10 in three axes. Hence, changes of the orientation, the movement, and the rotation of the bloodstream detector 10 can be calculated according to the values of the accelerations as well as the angular accelerations of the bloodstream detector 10 in three axes.

The signal output interface 500 is coupled to the light receiver 300 and the orientation sensor 400 for outputting a detection signal of the light receiver 300 and a detection signal of the orientation sensor 400. In one embodiment, the signal output interface 500 is a wireless communication interface. The wireless communication interface may be, for example, Bluetooth, zigbee, wireless network. In another embodiment, the signal output interface 500 is a wired communication interface. The wired communication interface may be, for example, serial peripheral interface (SPI) or general-purpose input/output (GPIO). The bloodstream detector 10 may be connected to external computer devices through the signal output interface 500. The detection signal of the light receiver 300 and the detection signal of the orientation sensor 400 are obtained through the signal output interface 500 for analyzing the brain bloodstream activities of the testee. Through the bloodstream activities of the testee, the brain activities of the testee can be estimated. For example, portions with frequent blood flowing indicate that the brain activities of the portions are more active.

As shown in FIG. 3, the bloodstream detector 10 may further comprise a signal preprocessing circuit 600 between the light receiver 300, the orientation sensor 400, and the signal output interface 500. The signal preprocessing circuit 600 is adapted to preprocess the detection signal of the light receiver 300 and the detection signal of the orientation sensor 400 for being outputted by the signal output interface 500. The signal preprocessing circuit 600 may comprise a filter for removing noise signals, an analog-to-digital converter, and other components. In one embodiment, the signal preprocessing circuit 600 may further calibrate the detection signal of the light receiver 300 according to the detection signal of the orientation sensor 400. In this embodiment, the signal calibration may be accomplished by using a Kalman filter or other recursive filters to remove interferences of noises generated by the movement or body-swing of the testee.

As shown in FIG. 4, the bloodstream detector 10 may further comprise a controlling unit 700. The controlling unit 700 is electrically connected to the light emitter 200 to control the light-emitting strength of one or more light emitter 200. Accordingly, the bloodstream detector 10 can provide lights with different light-emitting strengths for testees with different skin thicknesses or skin colors. In another embodiment, the timing and the duration (i.e., the light-emitting frequency) of the lights emitted by the light emitter 200 can be controlled. Therefore, the light emitter 200 can emit lights in a proper timing. The controlling unit 700 may be a microprocessor or other programmable control chips, or the controlling unit 700 may be a hardware circuit.

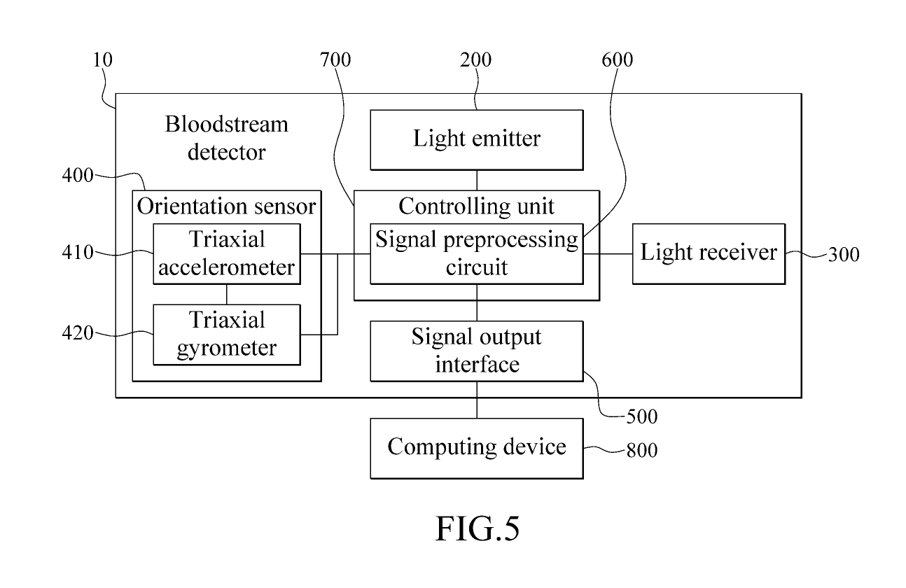

Please refer to FIG. 5. In one embodiment, the bloodstream detector 10 may be used with a computing device 800 as a bloodstream detecting device. The computing device 800 is electrically connected to the signal output interface 500 of the bloodstream detector 10 wiredly or wirelessly to receive the detection signal of the light receiver 300 and the detection signal of the orientation sensor 400, and the computing device 80 further calibrates the detection signal of the light receiver 300 according to the detection signal of the orientation sensor 400. Accordingly, as the signal processing manner of the signal preprocessing circuit 600 mentioned in the foregoing paragraphs, the noises of the detection signal of the light receiver 300 can be removed according to the values of the accelerations as well as the angular accelerations of the bloodstream detector 10 in three axes. Therefore, the computing device 800 can obtain optical signal accurately to obtain the information from the bloodstreams. In this embodiment, the computing device 800 may be a personal computer, a tablet, a cell phone, or other electronic devices with computing function.

As above, the bloodstream detector and the bloodstream detecting device of some embodiments of the instant disclosure can be manufactured with low costs and can be applied to the testees in a convenient manner. Moreover, the signals of the orientation sensor can optimize the signals of the light receiver, facilitating the reading of the signals of the light receiver.

What is claimed is:

1. A bloodstream detector, comprising:
a covering sheet comprising a detection surface;
at least one light emitter on the covering sheet and emitting lights outwardly from the detection surface;
at least one light receiver on the covering sheet and exposed from the detection surface to receive lights coming toward the detection surface;
an orientation sensor on the covering sheet to detect an orientation change of the bloodstream detector; and
a signal output interface coupled to the at least one light receiver and the orientation sensor to output a detection signal of the at least one light receiver and a detection signal of the orientation sensor.

2. The bloodstream detector according to claim 1, wherein the orientation sensor comprises at least one of a triaxial accelerometer and a triaxial gyroscope.

3. The bloodstream detector according to claim 1, wherein the signal output interface is a wireless communication interface.

4. The bloodstream detector according to claim 1, wherein the signal output interface is a wired communication interface.

5. The bloodstream detector according to claim 1, further comprising a controlling unit electrically connected to the at least one light emitter to control a light-emitting strength or a light-emitting frequency of at least one of the at least one light receiver.

6. The bloodstream detector according to claim 1, further comprising a signal preprocessing circuit between the at least one receiver, the orientation sensor, and the signal output interface to preprocess the detection signal of the at least one light receiver and the detection signal of the orientation sensor for being outputted by the signal output interface.

7. The bloodstream detector according to claim 6, wherein the signal preprocessing circuit calibrates the detection signal of the at least one light receiver according to the detection signal of the orientation sensor.

8. The bloodstream detector according to claim 1, wherein a wavelength of the lights emitted by the at least one light emitter is in a range between 750 nm and 860 nm.

9. The bloodstream detector according to claim 8, wherein the wavelength of the lights emitted by the at least one light emitter is in a range between 750 nm and 770 nm, in a range between 805 nm and 815 nm, in a range between 840 nm and 860 nm, or any combinations thereof.

10. The bloodstream detector according to claim 8, wherein the wavelength of the lights emitted by the at least one light emitter is in a range between 800 nm and 820 nm.

11. A bloodstream detecting device, comprising:
a bloodstream detector according to claim 1; and
a computing device electrically connected to the signal output interface of the bloodstream detector to receive the detection signal of the at least one light receiver and the detection signal of the orientation sensor and to calibrate the detection signal of the at least one light receiver according to the detection signal of the orientation sensor.

12. A bloodstream detector device, comprising:
a bloodstream detector according to claim 1; and
a computing device electrically connected to the signal output interface of the bloodstream detector to receive the detection signal of the at least one light receiver and the detection signal of the orientation sensor.
